(19) United States
(12) Patent Application Publication
(10) Pub. No.: US 2013/0184527 A1
(43) Pub. Date: Jul. 18, 2013

(54) Medical Instrument Light Source Connection Device

(71) Applicant: Omnimed Enterprises, LLC, Columbus, GA (US)

(72) Inventors: Sean A. Castellucci, Bradenton, FL (US); William M. Harper, Columbus, GA (US)

(73) Assignee: Omnimed Enterprises, LLC, Columbus, GA (US)

(21) Appl. No.: 13/674,988

(22) Filed: Nov. 13, 2012

Related U.S. Application Data
(63) Continuation-in-part of application No. 12/190,299, filed on Aug. 12, 2008, now abandoned.

(51) Int. Cl. A61B 1/00

(52) U.S. Cl. CPC A61B 1/00135
USPC 600/121

(57) ABSTRACT

A protector for a light conducting conduit constitutes an enlarged head attached to the distal end of the light conducting conduit. The enlarged head has an internal cavity with a shutter positioned adjacent its distal end. The shutter is opened by insertion of the light connection of a medical instrument into the internal cavity. The shutter closes when the light connection of the medical instrument is removed from the internal cavity. The enlarged head is made of a material with a low heat conductivity.

### CLAIM OF PRIORITY

This application is a continuation-in-part of copending U.S. patent application Ser. No. 12/190,299, filed Apr. 12, 2008, which is incorporated herein in its entirety.

### BACKGROUND OF THE INVENTION

1. Field of the Invention

The present invention relates to a device for protecting a light source conduit for a medical device.

2. Description of Related Art

Endoscopes are commonly used to replace more traditional invasive surgical procedures in contemporary medical and veterinary practice. The endoscope allows a surgeon to see images of the body's internal structures through natural openings or very small incisions. During traditional surgical procedures, incisions are required to be sufficiently large to allow direct visual observation of tissues and organs and to allow manipulation of the tissue and organs by a surgeon's hands and traditional surgical tools. During an endoscopic procedure, catheters and a small diameter endoscope tube are inserted into a natural opening in the body, or into a very small incision or puncture made in the body for purposes of the endoscopy. The endoscope tube, which may be rigid or flexible, not only provides an image for visual inspection and photography, but also enables the taking of biopsies and/or retrieval of foreign objects. An optical system contained in the endoscope tube transmits light or other electromagnetic waves from the body cavity and through the tube, enabling viewing of the interior of the body cavity through an eyepiece on the exterior of the body.

In some cases the endoscope tube is accompanied by a miniature camera that enables viewing of magnified images of the body's internal structures on an external screen.

In order to obtain a view of the interior of the body cavity, it is necessary to transmit the light or other radiation, such as infrared radiation, through the tube and into the body cavity, via a system of lenses and optical fibers, to illuminate the environment within the body cavity under observation. The illumination light and a return visual image of tissues and organs are conducted through the endoscope tube by optical fibers, enabling transmission of an image of the object under observation to the viewer. The illumination light is provided to the endoscope tube by an external light source, which is connected via a cable, typically a flexible optical-fiber cable, to the endoscope. One disadvantage associated with this arrangement is that extreme heating of the light source cable tends to occur in the vicinity where the cable connects to the endoscope. This heating, which is due in part to the high intensity of the lights typically used for endoscopy illumination purposes, undesirably presents a heat and/or fire hazard.

After completion of the operating procedure, the endoscope is typically detached from the light source cable, sometimes while the light source is still energized. Oftentimes, after such detachment, the end portion of the light source cable, which is typically extremely hot, is placed on a surface in the operating room. In some instances, that surface may be a flammable one, such as for example, the operating table drapes, bed sheets, surgical sponges and the like. In other instances, the end portion of the light source cable may be placed in the vicinity of the skin or clothing of the patient and/or operating personnel, as well as on or near a flammable material. This can result in fires, burning, singeing, or other damage to a variety of materials in the vicinity of the end portion of the light source cable. The risk and consequences of surgical fires are often increased by the presence of oxygen-rich environments often found under the drapes or in the surgical area due to the use of external oxygen sources.

A review of a number of information sources regarding surgical incidents, including medical literature, anecdotal reports, field investigations, and the United States Food and Drug Administration (F.D.A.) medical device problem reporting databases, indicates that in the United States up to 100 minor surgical fires occur annually, of which approximately ten are serious and one or two are fatal (M. E. Bruley, Surgical Fires: Perioperative Communication is Essential to Prevent This Rare But Devastating Complication, Quality and Safety in Health Care, December 2004; 13: 467-471). A search of the F.D.A.'s Manufacturer and User Device Experience Database, which can be found on the Internet at http://www.accessdata.fda.gov/scripts/cdrh/cfdocs/cfMAUDE/search.CFM, and the Medical Device Reporting Database, which can be found on the Internet at http://www.accessdata.fda.gov/scripts/cdrh/cfdocs/cfMDR/Search.CFM, reveals that from January 1995 to June 1998, there were 167 surgical fires, of which 56 (33%) were airway or oropharyngeal fires, 47 (28%) were fires on the head or neck, 40 (24%) were fires ignited on the outside of the patient and 24 (14%) were fires within the patient but not in the airway.

In the United Kingdom, the incidence of surgical fires related to flammable prepping agents and/or to high intensity endoscopic light sources has been of concern to the Medical Devices Agency, prompting it to issue alerts on the topic (The Patient is on Fire!: A Surgical Fires Primer, ECRI Institute Medical Device Safety Report, Guidance, January 1992; 21(1) 19-34; Fires From Oxygen Use During Head and Neck Surgery, ECRI Institute Medical Device Safety Report, Health Devices, Hazard Report, April 1995; 24(4): 155-157). These surgical fires and/or explosions, which can be severely disfiguring or fatal, warrant prevention.

Attempts have been made to reduce heating of the cable end portion, but many have proved ineffective or can be cumbersome. Such methods have included electronic devices to shut off or reduce the light from the light source when a disconnection from the endoscope is detected, and providing cooling air or fluid at the end of the cable.

U.S. Pat. Nos. 6,033,360 and 5,865,727 disclose a portable endoscope having a lamp as a light source that is enclosed within a lamp chamber casing. An armoring member is disposed around the lamp chamber casing, at a position where it is heated by heat radiated from a lamp light source in the portable endoscope. A heat insulating cover that is made of a material having a low thermal conductivity covers an outer surface of the armoring member.

U.S. Pat. No. 6,511,422, references suggestions of others of a retractable mechanical shroud that covers a light guide when not connected to an endoscope.

There remains a need in the art for an effective, efficient and easy-to-use method for preventing or reducing the heat and/or fire and explosion hazards presented by light source cables in an operating setting.

### BRIEF SUMMARY OF THE INVENTION

The present invention provides a device that prevents or reduces heat, fire and/or explosive hazards presented by light source cables when such cables are disconnected from a medical device such as an endoscope.

In one embodiment, the invention provides a protector for a light conducting conduit comprising at least one sheath surrounding or capable of surrounding at least a portion of the length of a distal end of the light conducting conduit; wherein the at least one sheath comprises a proximal end and a distal end; the sheath is capable of being connected to the light conducting conduit; the sheath and the light conducting conduit define a void extending longitudinally between the sheath and the light conducting conduit along at least a portion of a length of the sheath; and the sheath comprises a material having a low heat conductivity.

In another embodiment of the invention, a protector is provided for a light conducting conduit which comprises: (a) at least a first and a second sheath, wherein: (i) the first sheath comprises an inner wall, an outer wall, a proximal end and a distal end, wherein the first sheath is configured to surround at least a portion of a distal end of a light conducting conduit, and the first sheath is connected to or capable of being connected to a distal end of a light conducting conduit; and (ii) the second sheath comprises an inner wall and an outer wall, wherein the second sheath has a width measured in the transverse direction that is greater than a width of the first sheath and is configured so as to be placed around at least a portion of a length of the first sheath so as to define a void between the first sheath and the second sheath; and (b) a spring located in the void between the outer wall of the first sheath and the inner wall of the second sheath, wherein the second sheath is capable of being retracted and/or protracted.

The invention also includes an embodiment of a medical device assembly, comprising a medical device having a light conducting conduit and at least one sheath surrounding or capable of surrounding at least a portion of the length of a distal end of the light conducting conduit; wherein the at least one sheath comprises a proximal end and a distal end; the sheath is capable of being connected to the light conducting conduit; the sheath and the light conducting conduit define a void extending longitudinally between the sheath and the light conducting conduit along at least a portion of a length of the sheath; and the sheath comprises a material having a low heat conductivity.

In one embodiment of the medical device assembly there is a first sheath and a second sheath, wherein: the first sheath comprises an inner wall, an outer wall, a proximal end and a distal end, wherein the first sheath is configured to surround at least a portion of a distal end of the light conducting conduit, and the first sheath is connected to or capable of being connected to a distal end of the light conducting conduit; and the second sheath comprises an inner wall and an outer wall, wherein the second sheath has a width measured in the transverse direction that is greater than a width of the first sheath and is configured so as to be placed around at least a portion of a length of the first sheath so as to define a void between the first sheath and the second sheath; and a spring located in the void between the outer wall of the first sheath and the inner wall of the second sheath, wherein the second sheath is capable of being retracted and/or protracted.

The invention further includes embodiments of a method of treatment of a patient with a medical device having a light conducting conduit, wherein the method comprises using the medical device to operate on and/or treat a patient while applying a light source capable of generating heat; providing to the medical device at least one sheath surrounding or capable of surrounding at least a portion of the length of a distal end of the light conducting conduit; wherein the at least one sheath comprises a proximal end and a distal end; the sheath is capable of being connected to the light conducting conduit; the sheath and the light conducting conduit define a void extending longitudinally between the sheath and the light conducting conduit along at least a portion of a length of the sheath; and the sheath comprises a material having a low heat conductivity.

In another embodiment of the present invention, the protector at the distal end of the light conducting conduit comprises an enlarged head with an internal shutter. When the enlarged head is connected to a medical instrument, such as an endoscope, the shutter is opened by the engagement of the connector on the endoscope to the enlarged head. Once the endoscope has been disconnected from the enlarged head of the protector, the shutter closes to block the escape of light from the distal end of the light conducting conduit. Because the head of the protector is enlarged, the head can provide sufficient insulation between the distal end of light conducting conduit and the surrounding environment. The enlarged head of the protector may have various shapes including a cone shaped head and a rounded bulbous shaped head.

### BRIEF DESCRIPTION OF THE DRAWINGS

The foregoing summary, as well as the following detailed description of preferred embodiments of the invention, will be better understood when read in conjunction with the appended drawings. For the purpose of illustrating the invention, there are shown in the drawings a variety of embodiments of the invention. It should be understood, however, that the invention is not limited to the precise arrangements and instrumentalities shown in these drawings. In the drawings:

FIG. 1 is a partially broken, perspective view of a light conducting conduit having on its distal end a protector having an accordion-style sheath protracted around a distal end of a light conducting conduit, according to an embodiment of the present invention.

FIG. 2 is a partially broken, cross-sectional side view of the light conducting conduit and protector of FIG. 1, with the protector's accordion-style sheath retracted around the distal end of the light conducting conduit;

FIG. 3 is a partially broken, cross-sectional side elevation view of a light conducting conduit having on its distal end a stationary protector having a hinged cover over the distal end of the protector's sheath, according to a further embodiment of the present invention;

FIG. 4 is a partially broken, cross-sectional side elevation view of the light conducting conduit and protector of FIG. 3, with the hinged cover raised over the distal end of the protector's sheath;

FIG. 5 is a partially broken, cross-sectional side elevation view of a light conducting conduit having on its distal end a protector having a first sheath and a second sheath, where the second sheath is protracted around the distal end of a light conducting conduit, according to a further embodiment of the present invention; and

FIG. 6 is a partially broken, cross-sectional side elevation view of the light conducting conduit and protector of FIG. 5, with the protector's second sheath retracted around the distal end of the light conducting conduit.

FIG. 7 is a perspective view of the distal end of a light conducting conduit with an attached, conical-shaped protector in accordance with the present invention.

FIG. 8 is an exploded perspective view of the distal end of a light conducting conduit with an attached, conical shaped protector in accordance with the present invention.

FIG. 9 is a perspective view of the distal end of a light conducting conduit with an attached, rounded bulbous shaped protector in accordance with the present invention.

FIG. 10 is an exploded perspective view of the distal end of a light conducting conduit with an attached, rounded bulbous shaped protector in accordance with the present invention.

FIG. 11 is a perspective side-by-side view of the distal end of a light conducting conduit with an attached, conical shaped protector and of the distal end of a light conducting conduit with an attached, rounded bulbous shaped protector, both in accordance with the present invention.

FIG. 12 is a schematic cross-section view of the distal end of a light conducting conduit connected to a medical instrument by means of the conical shaped protector in accordance with the present invention.

FIG. 13 is a schematic cross-section view of the distal end of a light conducting conduit connected to a medical instrument by means of the bulbous shaped protector in accordance with the present invention.

FIGS. 14a-14e are respectively a perspective view, a top plan view, a front elevation view, a side elevation view, and a section view of a rounded bulbous shaped protector formed as a single piece in accordance with the present invention.

### DETAILED DESCRIPTION OF THE INVENTION

The protector of the invention is suitable for use with any medical device having light provided to it through a light conducting conduit, particularly such conduits that may be connected to a light source, for example a medical device such as an endoscope. Although the invention is particularly useful for non-portable medical devices, it may be used for portable medical devices as well. As used herein, “light conducting conduit” means any type of cord or cable capable of transmitting light from a light source to a medical or other device. The light conducting conduit has a proximal end and a distal end. As used herein, “proximal” and “distal” mean respectively the end of the light conducting conduit farthest from the patient and closest to the patient, and so also generally correspond herein respectively, to the end of the light conducting conduit which is closest to the light source (farthest away from the patient) and the end farthest from the light source (closest to the patient). As used herein, “end” means a portion of the conduit that forms the last part, lengthwise, of the conduit, and is not intended to mean only the extremity or “tip” of the conduit.

The protector is preferably located at the distal end of the light conducting conduit. The protector includes at least one sheath also having a proximal end and a distal end. The sheath is preferably generally circular in a transverse cross-sectionally configuration, however, other shapes such as oval, square, rectangular, and the like are within the scope of the invention. The internal width or internal diameter of the protector's sheath is larger than the outer width or diameter of the distal end of the light conducting conduit, thereby enabling the sheath to surround at least a portion of the length of the distal end of the light conducting conduit, and in some embodiments of the invention, enabling the distal end of the light conducting conduit to be connected to a medical device within the interior of the sheath.

The sheath may be connected to or capable of being connected to a light conducting conduit. The connection can be located anywhere along the length of the sheath, but is preferably located on either the proximal end or the distal end of the sheath, or alternatively on both such ends. The full extent of the sheath does not lie directly on the surface of the light conducting conduit. Rather, as noted above, a portion of the sheath is connected to the light conducting conduit, while the remainder of the sheath is separated from the light conducting conduit, and the inner surface of the sheath and the outer surface of the light conducting conduit define a void space therebetween. This configuration enables at least one of: movement of the sheath along at least a portion of the length of the distal end of the light conducting conduit, protraction and/or retraction of the sheath along at least part of the length of the distal end of the light conducting conduit, movement of the distal end of the light conducting conduit along at least part of the length of the sheath, or insertion of a medical device connection into the opening at the tip of the distal end of the sheath, and the like. By “tip” is meant herein the extremity or terminal part of the distal end of the sheath.

The protector may be manufactured as an extension of the light conducting conduit during manufacture of the conduit, thereby making it a connected and/or integral part of a light conducting conduit portion of a medical device and/or part of a medical device assembly. Alternatively, it may be connected to the light conducting conduit as an attachment or accessory subsequent to manufacture of the conduit and/or medical device, for example by welding, screwing, application of adhesive, quick-connect attachment, ferrule, socket, snap-fit attachment, or any other suitable method of connection or attachment. Detachable connections may be desirable where, for example, the protector is made to be retrofitted and/or a supplementary attachment to an existing light conducting conduit to provide the heat protection afforded by the present invention to already-existing medical device assemblies having such light conducting conduits.

Typical light conducting conduits have a generally cylindrical or tubular configuration. Thus, the sheath used in the protector herein is also preferably generally cylindrical or tubular, or it may have any other suitable configuration, as long as it is capable of surrounding at least a portion of the length of the distal end of the light conducting conduit. The sheath is formed so as to include a material which provides low heat conductivity, meaning that it is constructed of a material that prevents, substantially prevents, or reduces conduction of heat from the distal end of the light conducting conduit to the environment external to the sheath, thereby protecting external materials from heat or fire hazards. In a preferred embodiment of the invention, the heat that would be conducted from the distal end of the light conducting conduit to the environment external to the sheath, in the absence of the sheath, is reduced by the sheath by about 25% to about 100%, preferably about 45% to about 100%, more preferably about 60% to about 100%, and most preferably about 80% to about 100%. Examples of suitable materials for use in forming the sheath include low heat-conducting materials such as certain plastics, ceramics, and the like. It can also be formed of materials having a higher level of heat-conduction and also having a heat insulating material.

In one embodiment of the invention, the sheath may be retracted for connecting the distal end of the light conducting conduit to a medical device in a medical device assembly. Likewise, the sheath may be protracted when the medical device is disconnected from the light conducting conduit, thereby providing protection against the heat or fire hazard presented by the conduit. The sheath may also be arranged such that the retraction occurs automatically upon connection of a medical device to the conduit to form a medical device assembly and/or the protraction also occurs automatically upon disconnection of the medical device from the conduit. The protraction and/or retraction may be achieved manually or through use of a mechanism capable of causing the sheath to protract and/or retract. For example, the sheath may configured to have an accordion or telescoping design that may be manually or mechanically pushed up or down, automatically or not, to expose or protect the distal end of the conduit.

In a further embodiment of the invention, the sheath may enable protection of the distal end of the light conducting conduit while the sheath remains in a stationary position. For example, in this embodiment, the sheath may be attached to the conduit in a manner that maintains the sheath in a stationary position, while allowing the distal end of the conduit to be slid into the interior of the sheath for protection, or out of the sheath for connection to a medical device. Alternatively, in this embodiment, the inner wall of the stationary sheath may have a diameter that is large enough to enable the medical device to be connected to the distal end of the conduit within the interior of the sheath.

In an alternative embodiment of the invention, the sheath may have an isolation device at the tip of its distal end that is capable of causing the obstruction of at least a portion, and preferably the entirety, of the tip of the sheath's distal end, thereby isolating the distal end of the conduit from the environment external to the sheath. For example the isolation device may be a cover, cap, lid or other suitable device that can be caused to open or close, covering at least a portion, and preferably all, of the open area at the tip of the distal end of the sheath. Optionally, isolation may occur automatically upon the disconnection of the distal end of the conduit from a medical device. This isolation device may be attached to the sheath by any suitable method, such as for example by a hinge. The isolation device may be opened, for example, by pulling on a lip on a cover, applying pressure to a pressurized hinge, or any other suitable method. In one embodiment, the isolation device may be capable of sliding across the open area of the distal end of the sheath, preferably at the tip of the sheath.

In another embodiment of the invention, the protector may include multiple sheaths, all of which are stationary or retractable and/or protractable. In yet another embodiment of the invention, some of the sheaths may be stationary, while others are able to protract and/or retract. For example, the protector may include a first stationary sheath and a second retractable and/or protractable sheath. In this embodiment, the first sheath may be a stationary sheath having an inner wall, an outer wall, a proximal end, and a distal end. The first sheath may be connected to, or capable of being connected to, at least one point at the distal end of the light conducting conduit, enabling the first sheath to be firmly attached to the conduit. The inner wall of the first sheath has a width or diameter that is larger than that of the distal end of the light conducting conduit, so that the sheath may surround at least a portion of the length of the distal end of the light conducting conduit. In this embodiment, it is preferred that the first sheath be located such that it is not connected to the far extreme of the distal end of the conduit. Also in this embodiment of the invention, a second sheath is configured so as to be placed around at least a portion of the length of the first sheath, so as to define a void between the first sheath and the second sheath. The second sheath has an inner wall and an outer wall. The inner wall of the second sheath has a diameter or width, measured in the transverse direction that is greater than the diameter of the outer wall of the first sheath. In this embodiment, the second sheath may be retracted around the first sheath, allowing connection of a medical device to the distal end of the conduit. The protraction and/or retraction may be facilitated, for example by inclusion of a spring between the outer wall of the first sheath and the inner wall of the second sheath. The protraction and/or retraction, which may be performed manually or mechanically, may occur automatically upon disconnection and/or connection of the light conducting conduit from the medical device.

In another embodiment of the present invention, the protector at the distal end of the light conducting conduit comprises an enlarged head with an internal shutter. When the enlarged head is connected to a medical instrument, such as an endoscope, the shutter is opened by the engagement of the connector on the endoscope to the enlarged head. Once the endoscope has been disconnected from the enlarged head of the protector, the shutter closes to block the escape of light from the distal end of the light conducting conduit. Because the head of the protector is enlarged, the head can provide sufficient insulation between the distal end of the light conducting conduit and the surrounding environment. The enlarged head of the protector may have various shapes including a cone shaped head and a rounded bulbous shaped head.

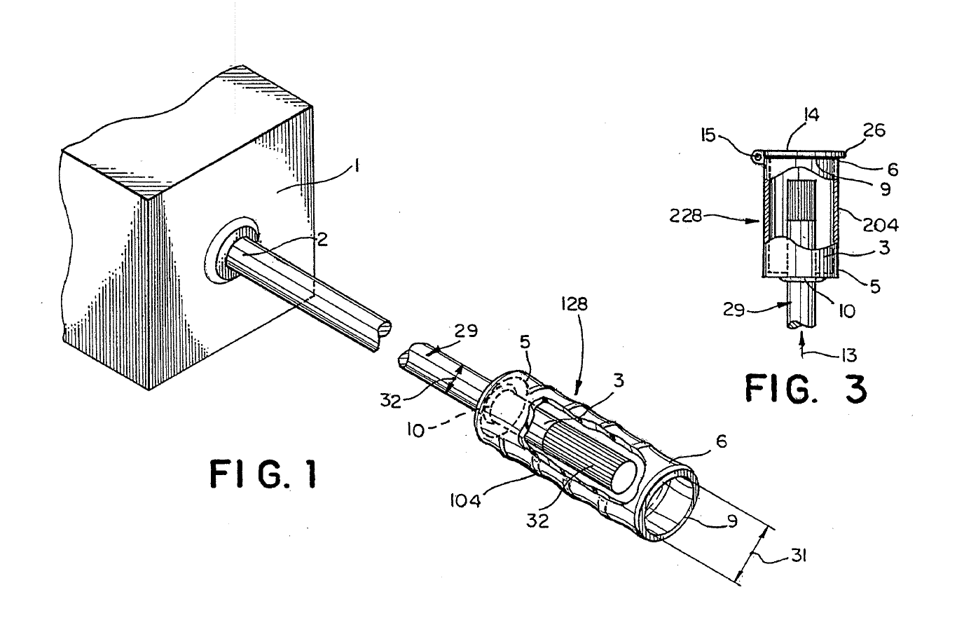

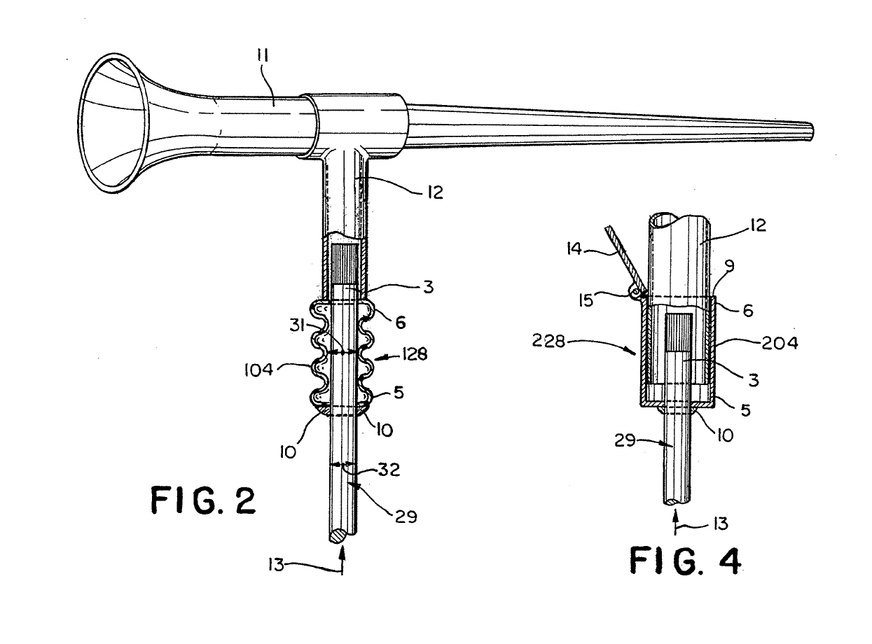

There is shown in FIGS. 1 and 2, a protector 128 according to one embodiment of the invention. In this embodiment, the protector 128 has an accordion-style sheath 104 that is capable of protracting and retracting around the distal end 3 of a light conduit 29. In FIG. 1, the sheath 104 of this embodiment is extended to the protracted position. In this position, the distal end 6 of the sheath 104 protects the distal end 3 of the light conducting conduit 29. The sheath 104 has a diameter 31 as measured in a transverse direction across the sheath that is wider than the outer diameter 32 of the distal end 3 of the light conduit 29, enabling the sheath 104 to surround at least a portion of the distal end 3 of the light conduit 29. The proximal end 5 of the sheath 104 is connected to the light conduit 29 at welded connection 10. Both the light conduit 29 and the sheath 104 have a generally tubular configuration. The tip 9 of the distal end 6 of the sheath 104 remains open to the environment.

The sheath 104 is preferably constructed of a material that prevents or substantially prevents conduction of the heat from the distal end 3 of the light conduit 29 to the environment external to the sheath 104, thereby protecting external materials, patients, and medical personnel from heat, explosion and/or fire hazard. The proximal end 2 of the light conduit 29 is connected to a light source 1 that is capable of providing a high intensity light suitable for use in medical procedures such as an endoscopy procedure.

FIG. 2, shows the accordion style sheath 104 of the same embodiment of the invention, but in the retracted position. The retracted sheath 104 enables the connection of the distal end 3 of the light conduit 29 to a light connection 12 on a medical device such as endoscope 11 shown herein for exemplary purposes.

FIGS. 3 and 4 show a protector 228 according to a different embodiment of the invention. In this embodiment, the protector 228 has stationary sheath 204 that is welded to the distal end 3 of a light conduit 29 at connection 10. In FIG. 3, the sheath 204 has a cover 14 at the tip 9 of the distal end 6 of the sheath 204. The cover 14 is in the closed position so that it isolates the tip 9 of the distal end 6 of the sheath 204 from the environment external to the sheath 204. The cover 14 has a lip 26 extending past the edge or outer diameter of the tip 9 of the distal end 6 of the sheath. 204. The cover 14 is connected to the sheath 204 by a spring-loaded hinge 15. As shown in FIG. 4, in this embodiment of the invention, the cover lip 26 can be manually lifted, causing the cover 14 to open. The open cover 14 enables the endoscope light connection 12 to be attached to the distal end 3 of the light conduit 29, within the interior of the sheath 204. When the endoscope light connection 12 is removed from the distal end 3 of the light conduit 29, the cover 14 automatically closes. While a spring-loaded hinge connection welded to the end of the distal end of the conduit is shown, it should be understood based on this disclosure that other methods of opening and closing the tip of the distal end to provide a cover are within the scope of the invention as are other methods for permanently or securely attaching the sheath 204 to the conduit 29.

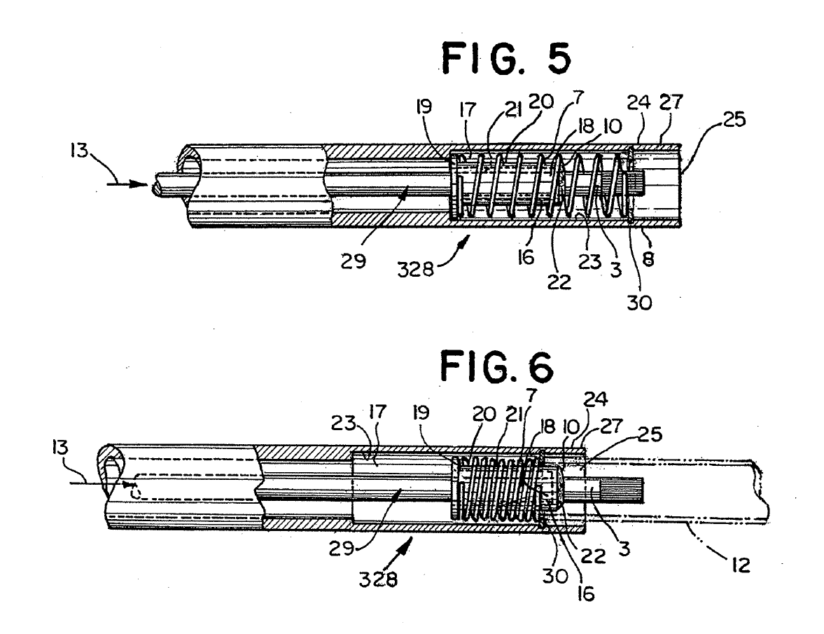

FIGS. 5 and 6 show a different protector 328 according to a yet another embodiment of the invention. As shown in FIGS. 5 and 6, the protector 328 has a stationary first sheath 16 and a retractable and protractable second sheath 27. In this embodiment, the first sheath 16 has an inner wall 20, an outer wall 21, a proximal end 17, and a distal end 18. The first sheath 16 is attached, preferably permanently or otherwise securely, to the conduit 29 at a point 10 at the tip 22 of the distal end 18 of the first sheath. 16. The inner wall 20 of the first sheath 16 defines an internal diameter that is greater than the outer diameter of the light conduit 29, so that the first sheath 16 surrounds at least a portion of the distal end 3 of the light conduit 29. The second sheath 27 has an inner wall 23 and an outer wall 24. The inner wall 23 of the second sheath 27 defines an internal diameter that is greater than the outer diameter defined by the outer wall of the first sheath 16, enabling the second sheath 27 to surround at least a portion of the distal end 18 of the first sheath 16, and at least a portion of the distal end 3 of the light conduit 29. A spring 7 is situated between the outer wall 21 of the first sheath 16 and the inner wall 23 of the second sheath 27. The spring 7 is held in place, for example, by a distal spring retainer 30, and a proximal spring retainer 19. In FIG. 5, the spring 7 is extended in its relaxed state, causing the second sheath 27 to be fully protracted, with the distal end 8 of the second sheath 27 surrounding the distal end 3 of the light conduit 29, enabling protection from the heat, explosion, and/or fire hazard presented by the distal end 3 of the light conduit 29. The tip 25 of the distal end 8 of the second sheath 27 is open, enabling insertion of a medical device, such as endoscope 11 into the second sheath 27 for connection with the distal end 3 of the light conduit 29. In FIG. 6, the spring 7 is compressed, causing the second sheath 27 to be in the retracted state. In this state, the endoscope connection 12 has been attached to the distal end 3 of the light conduit 29 in preparation for performance of an endoscopy procedure.

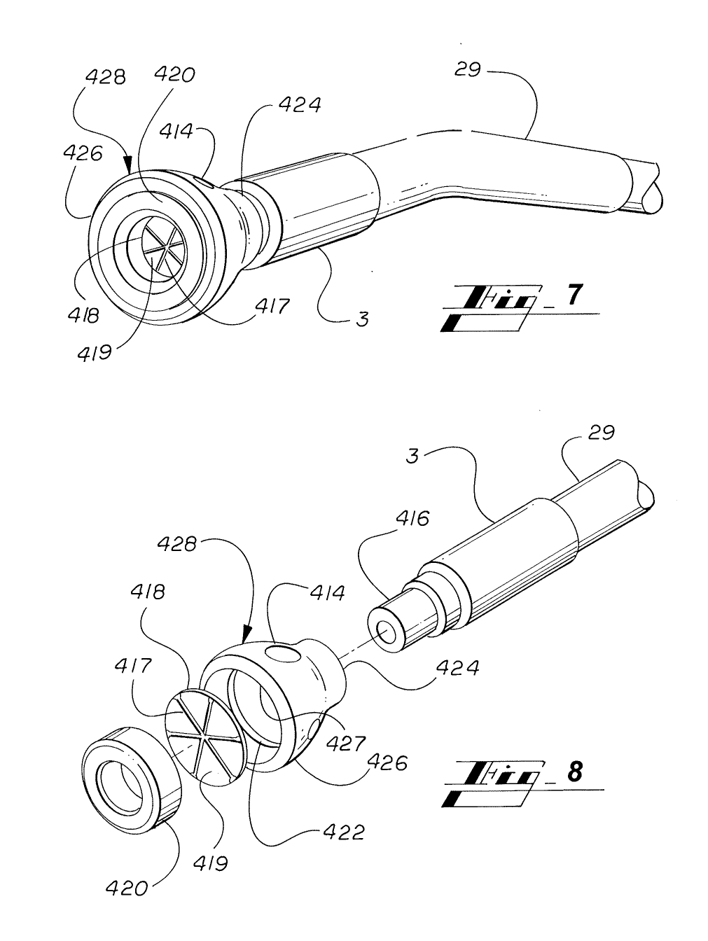

Turning to FIGS. 7-8 and 12, a protector 428 having an enlarged head 414 is attached to the distal end 3 of the light conducting conduit 29 by means of a connector 416. The enlarged head 414 is basically conical in shape having a narrow end 424 connected to the light conducting conduit 29 and a wide end 426 opposite from the narrow end 424. The enlarged head 414 also has an internal cavity 427 for receiving the light connection 12 of the endoscope 11. The enlarged head 414 is comprised of an insulating material that keeps heat generated in the internal cavity 427 by the light source 1 from being transferred to the surrounding environment. Such insulating materials include composites and silicon materials known to a person of ordinary skill in the art.

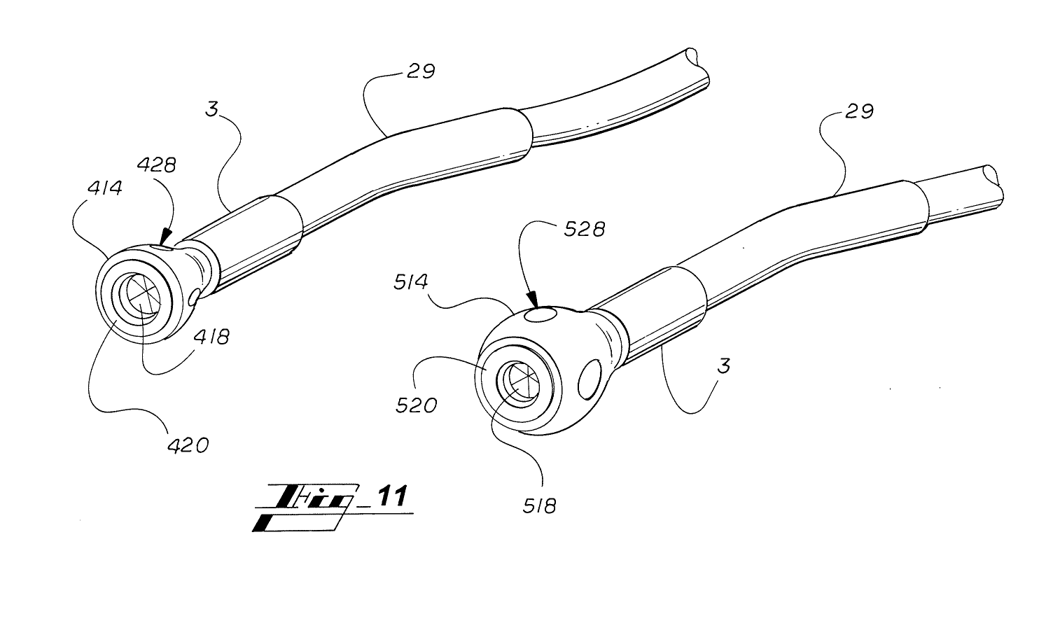

A flange 422 (FIG. 8) is located within the internal cavity 427 and adjacent the wide end 426 of the enlarged head 414. A disc-shaped shutter 418 is positioned against the flange 422 and held in place by means of a retaining ring 420. The shutter 418 is a membrane made of a flexible, heat resistant material known to persons of ordinary skill in the art. The shutter 418 is crisscrossed by radially extending slits 417 that end just short of the periphery of the shutter 418. The radially extending slits 417 create a series of pie-shaped flaps 419 that function as a shutter to open when the flaps 419 are displaced longitudinally with respect to the enlarged head 414 (FIG. 12) and to close as shown in FIGS. 7-8.

As shown in FIG. 12, the flaps 419 of the shutter 418 are displaced longitudinally when the endoscope light connection 12 is inserted into the internal cavity 427 of the enlarged head 414. Consequently, light from the light conducting conduit 29 is able to pass through the internal cavity 427, past the open shutter 418, and into the light connection 12 of the endoscope 11. When the endoscope light connection 12 is removed from the internal cavity 427 of the enlarged head 414, the flexible flaps 419 of the shutter 418 return to their original closed position as shown in FIGS. 7-8. With the shutter 418 in the closed position, the enlarged head 414 may safely come in contact with most surfaces in an operating room including surgical gowns, surgical drapes, sponges, and the like. Because the shutter is closed, the intense light from the light source 1 is precluded from impinging on surfaces and creating a fire hazard. In addition, because the head 414 of the protector 428 is enlarged around its wide end 426, the enlarged head 414 provides sufficient installation from the heat generated inside the internal cavity 427 to avoid a fire hazard to any surface with which the enlarged head 414 comes in contact.

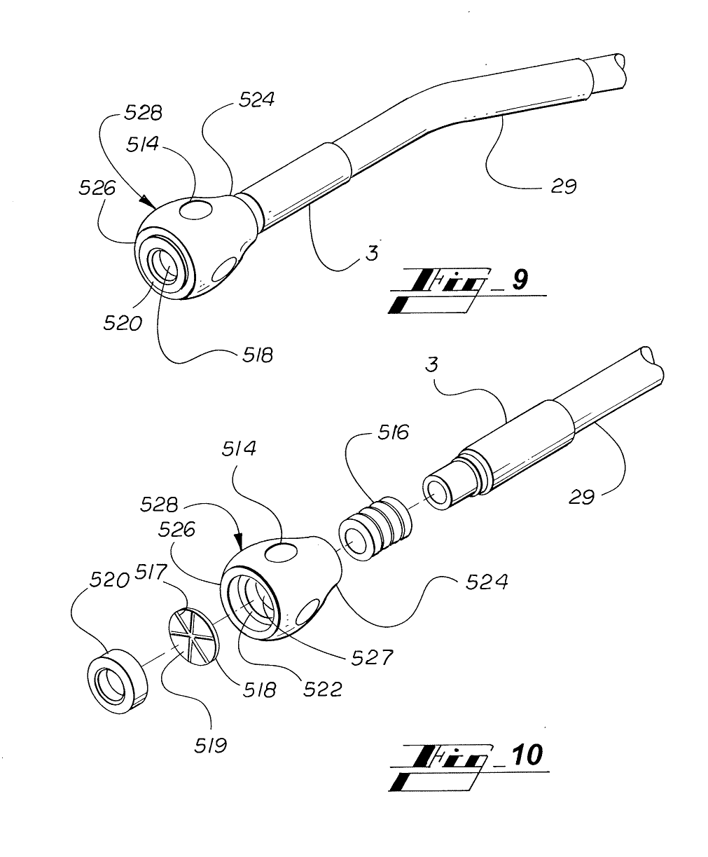

FIGS. 9-10 and 13 show a protector 528 which is similar to protector 428 described above except for the bulbous shape of the enlarged head 514. As shown in FIGS. 9-10, the enlarged head 514 is basically bulbous in shape having a narrow end 524 connected to the light conducting conduit 29 and a wide end 526 opposite from the narrow end 524. The enlarged head 514 also has an internal cavity 527 for receiving the light connection 12 of the endoscope 11. The enlarged head 514 is comprised of an insulating material that keeps heat generated in the internal cavity 527 by the light source 1 from being transferred to the surrounding environment. Such insulating materials include composites and silicon materials known to a person of ordinary skill in the art.

A flange 522 (FIG. 10) is a located within the internal cavity 527 and adjacent the wide end 526 of the enlarged head 514. A disc-shaped shutter 518 is positioned against the flange 522 and held in place by means of a retaining ring 520. The shutter 518 is a membrane made of a flexible, heat resistant material known to a person of ordinary skill in the art. The shutter 518 is crisscrossed by radially extending slits 517 that end just short of the periphery of the shutter 518. The radially extending slits 517 create a series of pie-shaped flaps 519 that function as a shutter to open when the flaps 519 are displaced longitudinally with respect to the enlarged head 519 (FIG. 12) and to close as shown in FIGS. 9-10.

As shown in FIG. 12, the flaps 519 of the shutter 518 are displaced longitudinally when the endoscope light connection 12 is inserted into the internal cavity 527 of the enlarged head 514. Consequently, light from the light conducting conduit 29 is able to pass through the internal cavity 527, past the open shutter 518, and into the light connection 12 of the endoscope 11. When the endoscope light connection 12 is removed from the internal cavity 527 of the enlarged head 514, the flexible flaps 519 of the shutter 518 return to their original closed position as shown in FIGS. 9-10. With the shutter 518 in the closed position, the enlarged head 514 may safely come in contact with most surfaces in an operating room including surgical gowns, surgical drapes, sponges, and the like. Because the shutter is closed, the intense light from the light source 1 is precluded from impinging on surfaces and creating a fire hazard. In addition, because the head 514 of the protector 528 is enlarged around its wide end 526, the enlarged head 514 provides sufficient installation from the heat generated inside the internal cavity 527 to avoid a fire hazard to any surface on which the enlarged head 514 comes in contact.

FIG. 11 shows a side-by-side comparison of the protector 428 with its conical shape and the protector 528 with its bulbous shape.

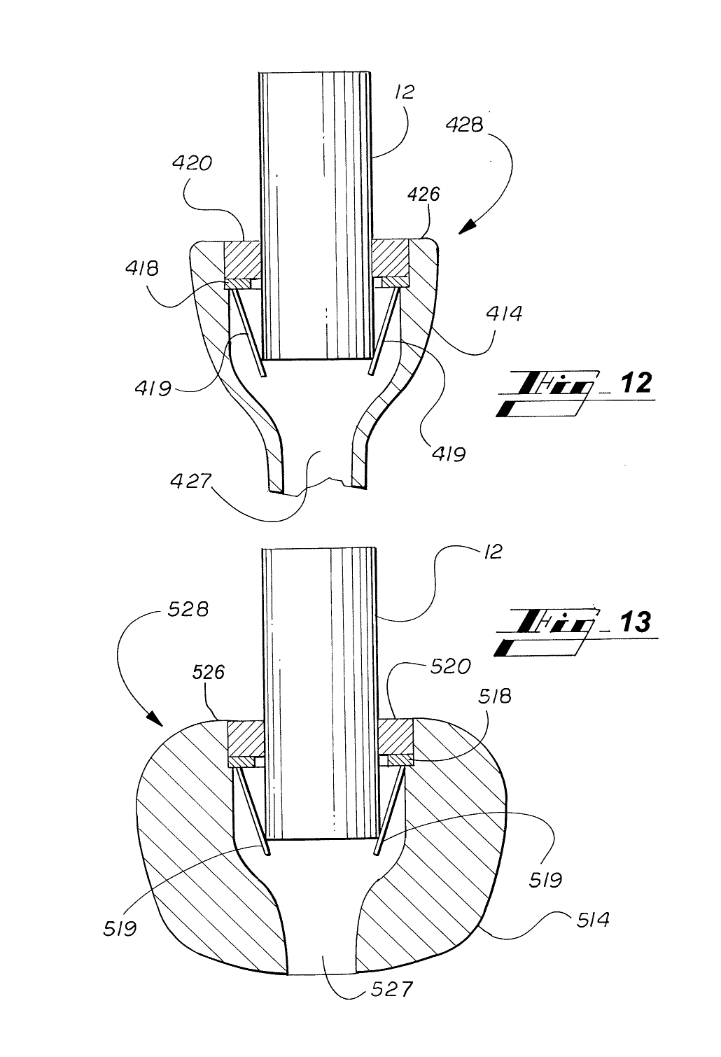

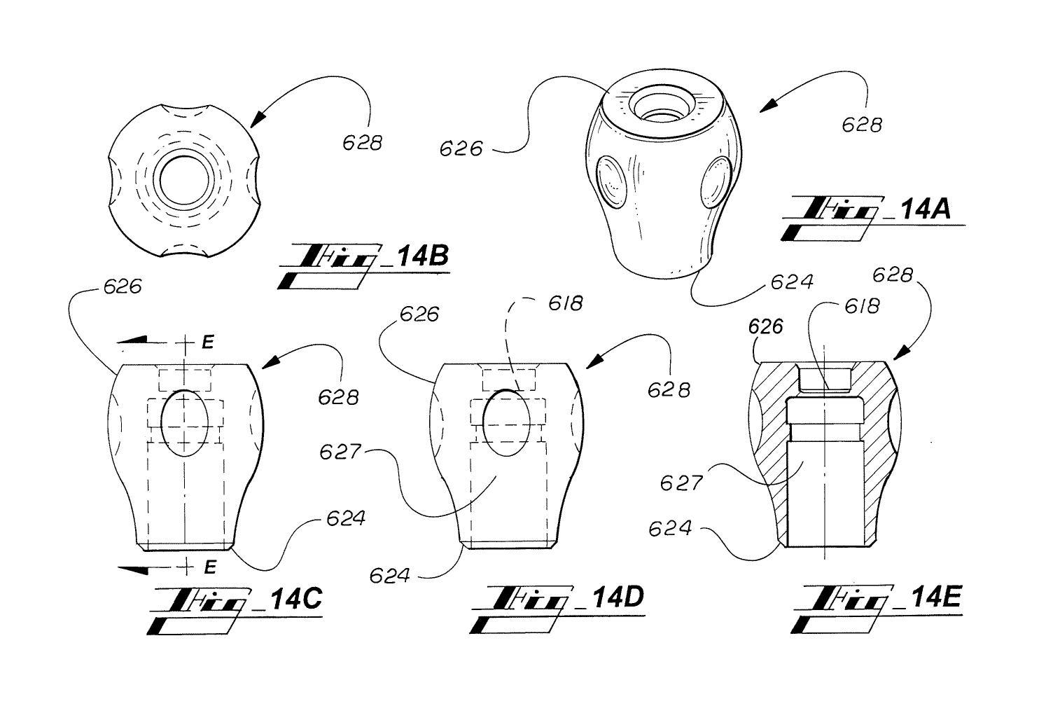

FIGS. 14a-14e disclose a single piece molded protector 628. The protector 628 has a bulbous shape and has an internal cavity 627 for receiving the light connection 12 of the endoscope 11. The protector 628 has a shutter 618 (as previously described with respect to shutters 418 and 518) for opening the internal cavity 627 at the wide end 626 when the light connection 12 of the endoscope 11 is connected to the protector 628 and for closing the internal cavity 627 at the wide end 626 when the light connection 12 of the endoscope 11 is disconnected from the protector 628. The protector 628 is molded from silicon rubber or other suitable thermoplastic materials as a single piece with the integral shutter 618.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims and the foregoing description of the invention.

1. A protector for a light conducting conduit with a proximal end and a distal end, the protector comprising
a. an enlarged head having
i. a proximal end for attachment to the distal end of light conducting conduit;
ii. a distal end for engaging a light connection of a medical instrument;
iii. an internal cavity extending between the proximal end and a distal end of the enlarged head; and
iv. a shutter positioned adjacent the distal end of the enlarged head,
wherein the shutter is opened by insertion of the light connection of the medical instrument into the internal cavity, wherein the shutter closes when the light connection of the medical instrument is removed from the internal cavity, and wherein the enlarged head comprises a material having a low heat conductivity.

2. The protector according to claim 1, wherein the shutter is integrally formed with the enlarged head.

3. The protector according to claim 1, wherein the shutter comprises a membrane with radially extending crisscrossed slits that defining pie shaped flaps.

4. The protector according to claim 1, wherein the enlarged head is conical in shape.

5. The protector according to claim 7, wherein the enlarged head is bulbous in shape.
